(19) United States
(12) Patent Application Publication
(10) Pub. No.: US 2021/0396703 A1
(43) Pub. Date: Dec. 23, 2021

(54) Rapid Electrochemical Point-of-Care COVID-19 Detection in Human Saliva

(71) Applicant: Northeastern University, Boston, MA (US)

(72) Inventors: Sheyda NAZARIAN, Boston, MA (US); Nadia FARSAEIVAHID, Jamaica Plain, MA (US); Christian GRENIER, Medford, MA (US); Yunqing DU, Quincy, MA (US); Ming L. WANG, Ipswich, MA (US)

(21) Appl. No.: 17/351,211

(22) Filed: Jun. 17, 2021

Related U.S. Application Data
(60) Provisional application No. 63/040,152, filed on Jun. 17, 2020.

(51) Int. Cl. G01N 27/327; G01N 27/30

(52) U.S. Cl. CPC G01N 27/3275; G01N 27/308

(57) ABSTRACT

Novel devices and methods for rapid, liquid-based detection of viruses are provided, including rapid saliva-based detection of SARS-CoV-2. The devices can include one or more layers of graphene, and/or carbon nanotubes, nanoparticles, cross-linkers, redox probes, ACE2 enzyme, and aptamer or antibody (including SARS-CoV-2 antibody). Forward, and optionally reverse waveforms can be applied to the devices to provide voltammograms indicative of positive or negative detection of viruses.

### CROSS-REFERENCE TO RELATED APPLICATIONS

This application claims priority to U.S. Provisional Application No. 63/040,152, filed 17 Jun. 2020, which is incorporated by reference herein in its entirety.

### BACKGROUND

During the COVID-19 pandemic, rapid viral detection has become increasingly important. Rapid viral detection is vital to hindering the spread of disease during pandemics such as COVID-19. Existing detection methods each have significant limitations. PCR-based tests are non-invasive (using nasal swabs or saliva) but must be shipped off to molecular biology labs for testing. Antibody tests are relatively fast point-of-care (POC) tests, but often require blood samples and are semi-quantitative. The important and unsolved issue in pandemics such as COVID-19 is being able to provide viral tests to the highest number of individuals, including asymptomatic patients.

### SUMMARY

The present technology provides saliva-based POC biosensors that utilize nanotechnology to yield a non-invasive, rapid, and accurate POC viral detection device and method.

An aspect of the technology is an electrochemical sensor device for detection of a virus in a fluid sample. The device includes a substrate, a working electrode and a counter electrode disposed on the substrate, and a sample placement area on the substrate. The sample placement area is configured for deposition of a fluid sample into the sample placement area, such that the deposited fluid sample covers the working and counter electrodes. One or more functionalization layers is deposited on a surface of the working electrode. The surface is configured for exposure to the fluid sample deposited in the sample placement area. Each functionalization layer comprises one or more sensor elements, metallic nanoparticles, a crosslinker, a redox reagent, and a virus receptor.

Another aspect of the technology is a system for electrochemical detection of a virus. The system includes the electrochemical sensor device described above, a power source capable of supplying a desired voltage signal to the electrodes of the device, and a detection circuit capable of detecting current between electrodes of the device.

Yet another aspect of the technology is a method of detecting a virus in a fluid sample. The method includes depositing the sample into the sample placement area of the sensor device described above, as part of the system described above. A voltage signal, such as a ramped series of square wave voltage pulses, is applied to electrodes of the sensor device, and the resulting current is measured. In the presence of virus in the fluid sample that binds to the virus receptor of the sensor device, current is reduced in proportion to the concentration of the virus in the fluid sample.

The present technology can be further summarized by the following list of features.

1. An electrochemical sensor device for detection of a virus in a fluid sample, the device comprising:

a substrate;

a working electrode and a counter electrode disposed on the substrate;

a sample placement area on the substrate, the sample placement area configured for deposition of a fluid sample into the sample placement area, wherein the deposited fluid sample covers the working and counter electrodes;

one or more functionalization layers deposited on a surface of the working electrode, the surface configured for exposure to the fluid sample deposited in the sample placement area; wherein each functionalization layer comprises one or more sensor elements, metallic nanoparticles, a crosslinker, a redox reagent, and a virus receptor.

2. The electrochemical sensor device of feature 1, wherein the device comprises from 2 to about 10 stacked functionalization coating layers.
3. The electrochemical sensor device of feature 1 or 2, wherein the one or more sensor elements, metallic nanoparticles, polymer, redox reagent, and virus receptor of each functionalization layer are disposed in separate sublayers of the functionalization layers.
4. The electrochemical sensor device of feature 3, wherein the sublayers of each functionalization layer are disposed in the order (from electrode surface upwards): sensor elements, metallic nanoparticles, crosslinker, redox reagent, virus receptor.
5. The electrochemical sensor device of feature 1 or 2, wherein the one or more sensor elements, metallic nanoparticles, polymer, redox reagent, and virus receptor of each functionalization layer are present in a single continuous functionalization layer.
6. The electrochemical sensor device of feature 5, wherein the one or more functionalization layers are each produced by drop casting.
7. The electrochemical sensor device of any of the preceding features, wherein the sensor elements comprise a material selected from the group consisting of carbon nanotubes, graphene, graphene oxide, fullerene, buckypaper, graphite, carbon nanofibers, carbon nanowires, carbon nanopowder, nanorods, quantum dots, and mixtures thereof.
8. The electrochemical sensor device of feature 7, wherein the sensor elements comprise a mixture of carbon nanotubes and graphene.
9. The electrochemical sensor device of any of the preceding features, wherein the metallic nanoparticles comprise a metal selected from the group consisting of gold, silver, platinum, palladium, iron, iron oxide, copper, zinc, titanium, rhodium, ruthenium, rhenium, and mixtures thereof.
10. The electrochemical sensor device of any of the preceding features, wherein the crosslinker is selected from the group consisting of NHS.EDC, streptavidin, 1,6-hexanedithiol, glutaraldehyde, cysteamine, alkanethiol, protein A, 3-aminopropyltriethoxysilane (APTES), sulfosuccinimidyl 4-(N-maleimidomethyl) cyclohexane-1-carboxylate), dithiobis (succinimidyl propionate) (DSP), sulfhydryl-reactive (maleimide), carboxy-PEG-amine or -carboxylic acid, carboxy-PEG-lipoamide (bidentate thiol), carboxy-PEG-thiol or -carboxylic acid, methyl-PEG-amine, methyl-PEG-lipoamide (bidentate thiol), Protein G, disuccinimidyl suberate (DSS), and combinations thereof.
11. The electrochemical sensor device of any of the preceding features, wherein the redox reagent is selected from the group consisting of methylene blue, thionine, ferrocyanide/ferricyanide, horseradish peroxidase, glucose oxidase, alkaline phosphatase, rrease, β3-galactosidase, and combinations thereof.
12. The electrochemical sensor device of any of the preceding features, further comprising a reference electrode disposed in the sample placement area, wherein the deposited fluid sample covers the reference electrode.
13. The electrochemical sensor device of any of the preceding features, where any of the electrodes comprises a conductive material selected from the group consisting of gold, graphite, carbon, platinum, silver/silver chloride, aluminum, graphene, lead, carbon steel, nickel, chromium, copper, silver, tantalum, antimony, tin, tungsten, iridium, bismuth, titanium, molybdenum, and combinations thereof.
14. The electrochemical sensor device of any of the preceding features, wherein the substrate comprises a material selected from the group consisting of plastic, glass, polymer, silicon, stainless steel, polyimide, polyester, polydimethylsiloxane (PDMS), paper, polyethylene terephthalate, polyethylene naphthalate, cotton, poly(methylmethacrylate), and combinations thereof.
15. The electrochemical sensor device of any of the preceding features, wherein the one or more functionalization layers further comprise a surfactant selected from the group consisting of tergitol, sodium dodecyl sulfate (SDS), Triton X-100, Tween 20, Tween 80, polyethylene glycol (PEG), pluronic, polysorbate 80, polysorbate 20, cetyltrimethylammonium bromide, CTAB, (3-((3-cholamidopropyl) dimethylammonio)-1 propanesulfonate) (CHAPS), Span 60, lauric acid, oleic acid, digitonin, decyl glucoside, poloxamer, n-octyl-β-D-thioglucopyranoside, n-dodecyl-β-D-maltoside, lauryl glucoside, ethoxylated amides, alkyl polyglycosides, carbohydrate-derivate ethoxylates, and combinations thereof.
16. The electrochemical sensor device of any of the preceding features, wherein the one or more functionalization layers further comprise a surface blocking agent selected from the group consisting of bovine serum albumin (BSA), chicken serum albumin (CSA), 11-mercapto 1-undecanol (MCU), polyethylene glycol (PEG/HS-PEG), Tween 20, fish gelatin, non-fat dry milk, casein, whole sera, polyvinyl alcohol (PVA), polyvinylpyrrolidone (PVP), and combinations thereof.
17. The electrochemical sensor device of any of the preceding features, wherein the virus receptor is selected from the group consisting of cell surface proteins and fragments and variants thereof, antibodies and fragments thereof, aptamers, and combinations thereof.
18. The electrochemical sensor device of any of the preceding features, wherein the device is configured for detecting a virus selected from the group consisting of HIV-1, HIV-2, Zika virus (ZIKV), SARS-CoV, SARS-CoV-2, and combinations thereof.
19. The electrochemical sensor device of feature 18, wherein the virus is SARS-CoV-2.
20. The electrochemical sensor device of feature 19, wherein the virus receptor is selected from the group consisting of antibodies and fragments thereof binding to SARS-CoV-2 spike protein, envelope protein, RBD protein, or nucleocapsid protein; angiotensin-converting enzyme 2 (ACE2), SARS-CoV-2 specific aptamers, CR3022 antibody, CR3009 antibody, CR0018 antibody, and combinations thereof.
21. The electrochemical sensor device of any of the preceding features, wherein the device is configured for use in performing an electrochemical measurement comprising square wave voltammetry (SWV), amperometry, cyclic voltammetry (CV), or electrochemical impedance spectroscopy (EIS).
22. The electrochemical sensor device of any of the preceding features, wherein the device is configured for detecting a virus in a fluid sample selected from the group consisting of saliva, urine, tears, sweat, blood, and interstitial fluid.
23. The electrochemical sensor device of any of the preceding features, wherein the device is capable of detecting virus in a fluid sample down to a virus concentration of at least about 106 copies/mL.
24. The electrochemical sensor device of any of the preceding features, wherein the device is capable of detecting virus in a fluid sample down to a virus concentration of at least about 10⁵ copies/mL.
25. The electrochemical sensor device of any of the preceding features, wherein the device is capable of detecting virus in a fluid sample down to a virus concentration of at least about 10⁴ copies/mL.
26. The electrochemical sensor device of any of the preceding features, wherein the device comprises a filter for pre-treatment of a deposited fluid sample.
27. The electrochemical sensor device of any of the preceding features, wherein the device is configured for detecting SARS-CoV-2 down to a concentration of at least about 10⁵ copies/mL of a fluid sample; wherein the device comprises ACE2 and an antibody to S1 spike protein antibody as virus receptors; wherein the device comprises carbon nanotubes and graphene as sensor elements; wherein the device comprises gold nanoparticles; and wherein the device comprises thionine as redox reagent.
28. A system for electrochemical detection of a virus, the system comprising the electrochemical sensor device of any of the preceding features, a power source capable of supplying a desired voltage signal to the electrodes of the device, and a detection circuit capable of detecting current between electrodes of the device.
29. The system of feature 28, further comprising a transmitter for sending a wireless signal from the system to a computer, mobile phone, remote monitoring device, or network.
30. A method of detecting a virus in a fluid sample, the method comprising depositing the sample into the sample placement area of the device of any of features 1-27 or the system of feature 28 or 29, applying a voltage signal to electrodes of the device, and obtaining a measure of the presence, absence, amount, or concentration of the virus in the fluid sample.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1A shows a schematic illustration of a cross section of a working electrode according to the present technology. Substrate 1 is coated with a layer of sensor elements 2, which in turn is coated with a layer of metallic nanoparticles 3. The top layer is a functionalization coating 7 that includes a crosslinking reagent, a redox reagent, and a virus receptor, which can be present as separate sublayers (not shown) or as an essentially homogeneous mixture. In some embodiments, components of one layer may mix to some extent with adjacent layers; in other embodiments they remain separate. FIG. 1B shows an embodiment of a working electrode in which functionalization coating 7 contains sublayers of crosslinker 4, redox reagent 5, and virus receptor 6. FIG. 1C illustrates a multilayered embodiment in which multiple functionalization coating layers 7 are stacked one above the other. Each functionalization coating layer 7 can be subdivided into sublayers 4, 5, and 6 as shown in FIG. 1B, or the components can be mixed to form an essentially homogeneous layer. The upper functionalization layers 7 also can optionally repeat the sensor element layer 2 and/or the metallic nanoparticle layer 3 as shown in FIG. 1A.

FIG. 2A is a schematic illustration of an ACE2 enzyme-based, SARS-CoV-2 sensor that binds virus through its binding sites to ACE2. FIG. 2B is a schematic illustration of a SARS-CoV-2-specific antibody-based sensor that binds virus through its binding sites to the antibodies. FIG. 2C is a schematic illustration of a sensor using the combination of ACE2 enzyme and SARS-CoV-2 antibody that binds virus through its different binding sites, to both ACE2 and antibody, leading to lower risk of false negative and false positive readouts.

FIG. 3A depicts a staircase wave component of a square wave voltammetry excitement potential, where every period on the staircase is increased by a predetermined parameter Eₛₜₑₚ. FIG. 3B depicts a square wave component of the square wave voltammetry excitement potential, where the amplitude of the wave is a predetermined parameter Eₐₘₚ, and the period is identical to that of the staircase wave. FIG. 3C depicts a combination of the component waves (staircase wave component and square wave component) that produce the square wave voltammetry excitement waveform.

FIG. 4A depicts a calculated example of forward (Ψ_{F}, bottom waveform), reverse (Ψ_{R}, middle waveform) and total (Ψ_{T}, top waveform) waveforms that make up the final voltammogram. This is a graphical representation and does not depict an actual result. FIG. 4B depicts calculated examples of final output voltammograms with a predicted positive result waveform (bottom waveform) when virus is present in the solution, versus when virus is not present (negative result is top voltammogram). An example threshold value for positive and negative results is illustrated as the dotted horizontal line. This is a graphical representation and does not depict an actual result.

FIG. 5A shows a measured calibration curve of sensor responses to different concentrations of SARS-CoV-2 pseudovirus. FIG. 5B shows square wave voltammetry test results performed at 20 Hz frequency and 0.5 V amplitude in the voltage range of 0 to −0.7 volts using a functionalized electrode sensor to detect the indicated concentrations of SARS-CoV-2 pseudovirus. FIG. 5C shows results from a negative control test using a sensor whose working electrode was functionalized with a mouse IgG antibody that does not bind to SARS-CoV-2 pseudovirus.

### DETAILED DESCRIPTION

The present technology provides a convenient and extremely rapid point of care (POC) test for detection of virus in infected humans or other animals using a fluid sample, including saliva. This test could help address the global challenge of providing rapid pathogen detection to stop the spread of emerging viruses such as SARS-CoV-2 and its variants, as well as bacterial pathogens and other microbial pathogens having known receptors on host cells or available antibodies or aptamers with specificity and affinity sufficient to bind the pathogen to the sensor's functionalization coating. The POC test can analyze a liquid sample from a human or other animal subject and provide immediate or very rapid indication of the presence of a virus above a threshold concentration. In a multiplex format, the test can provide information as to the presence above a threshold concentration or absence at a threshold concentration of two or more viruses or virus variants simultaneously. The saliva based POC biosensor utilizes nanotechnology and robust, cross-linked components, yielding non-invasive, rapid, and accurate POC viral detection tests and kits with long shelf life. The results can be obtained in 3-5 minutes for COVID-19 detection using saliva. The rapidity of the test makes it an ideal option to screen for infected individuals (including asymptomatic individuals), at hospitals, clinics and health care facilities, workplaces, airports, ports of entry, schools, in community screening settings, in the home, and for people on the go, even during travel. Furthermore, the device can be provided in an affordable home viral test kit. The device is non-invasive, and the possibility of self-collection reduces the risk of contamination for healthcare workers.

While the present technology can be applied to any liquid sample, saliva contains plentiful disease biomarkers, including virus particles and portions thereof, which can be measured in order to detect and diagnose a variety of diseases. Saliva analysis is emerging as a non-invasive substitute to blood, serum, plasma, and urine analyses in diagnostic sampling. For example, saliva has been used to detect HIV¹, hepatitis A², B³ and C,^{4,5} oral cancer,⁶⁻⁸ breast cancer,^{8,9} pancreatic cancer,^{8,10} lung cancer,^{11,12} cardiovascular disease,¹³ and diabetes¹⁴. Saliva also demonstrates great potential as a diagnostic fluid for HIV-1 and HIV-2,¹⁵ Zika virus (ZIKV),^{16,17} SARS-CoV¹⁸ and SARS-CoV-2^{19,20} within the first week of symptom onset.

Furthermore, when testing saliva and nasopharyngeal aspirate (NPA) in parallel for the respiratory viruses, respiratory syncytial virus and influenza, the results had a 93% agreement, and the reported sensitivity and specificity for saliva and NPA are 90.8% and 100%, respectively.²¹ With some viruses like Zika virus, viral load in saliva is actually higher than in blood.¹⁷ In a study conducted at Hong-Kong University, saliva had a higher detection rate for COVID-19 in the early stages of infection before lung lesions develop,^{18′19} while in some patients coronavirus was detected in saliva, but not in nasopharyngeal specimen.¹⁹ Moreover, it is reported that the concentration of SARS-CoV-2 is higher in saliva than in blood or urine.²⁰

In view of the great potential of saliva specimens, the Food and Drug Administration (FDA) has approved saliva as a reliable and valid specimen for COVID-19 diagnosis in a test developed by Rutgers University.²² This is important, as self-collection of saliva minimizes the chance of exposing healthcare workers to the virus since saliva samples can be collected outside of hospitals, without the surveillance of trained healthcare workers.

As discussed above, a critical issue in pandemics such as COVID-19 is the ability to provide an accurate test to the highest number of individuals, including asymptomatic patients. In an example, an accurate test could be rapidly deployed at a low cost, or freely distributed, to individuals who want to utilize the accurate test. The present technology successfully addresses these issues and can provide real-time POC testing systems. A real-time POC testing system can play a crucial role in effective disease control and appropriate therapeutic intervention. This detection system offers patients a simple, non-invasive, and rapid test wherein the user can perform the test by him/herself at home, laboratory, or any location, without the surveillance of trained personnel. To achieve this goal, the proposed biosensor is used in combination with an electronic meter to conduct an electrochemical measurement of SARS-CoV-2 concentration.

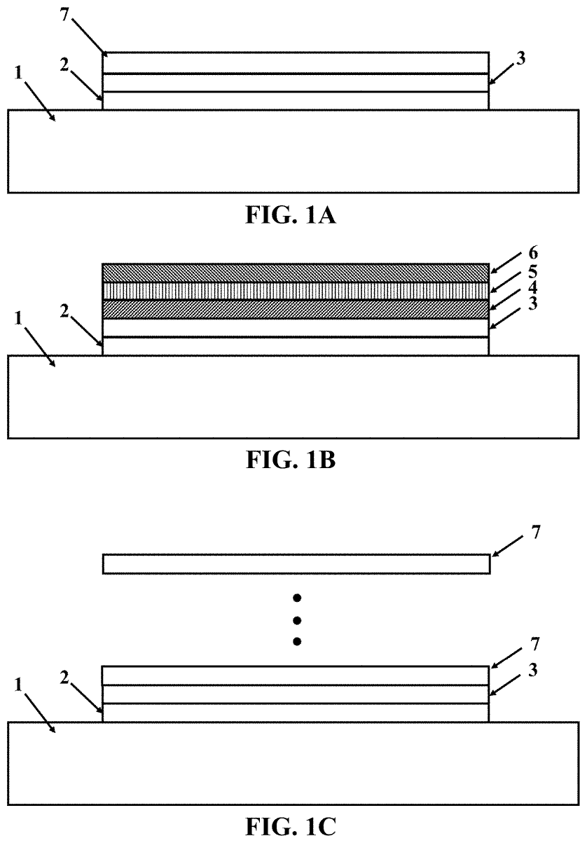

The key aspects of the electrochemical biosensor device are the composition and structure of the functionalization coating applied to the working electrode of the device. General schemes for the functionalization coating and structure of the electrode are shown in FIGS. 1A-1C. As shown in FIG. 1A, substrate 1, which is electrically insulating or semiconducting, is coated in the sample placement area with a layer of sensor elements 2, such as carbon nanotubes and/or graphene flakes. The sensor elements in turn are coated with a layer of conductive metallic nanotubes 3. The nanotube layer is then overlaid with one or more functionalization layers 7. As shown in FIG. 1C, two or more functionalization layers 7 may be stacked for increased sensitivity, as needed. In the embodiment shown in FIG. 1B, the functionalization layer can be in turn divided in to sublayers, such as a layer of crosslinker 4, a layer of redox reagent 5, and a layer of virus receptor 6, which is exposed to the sample fluid deposited in the sample placement area.

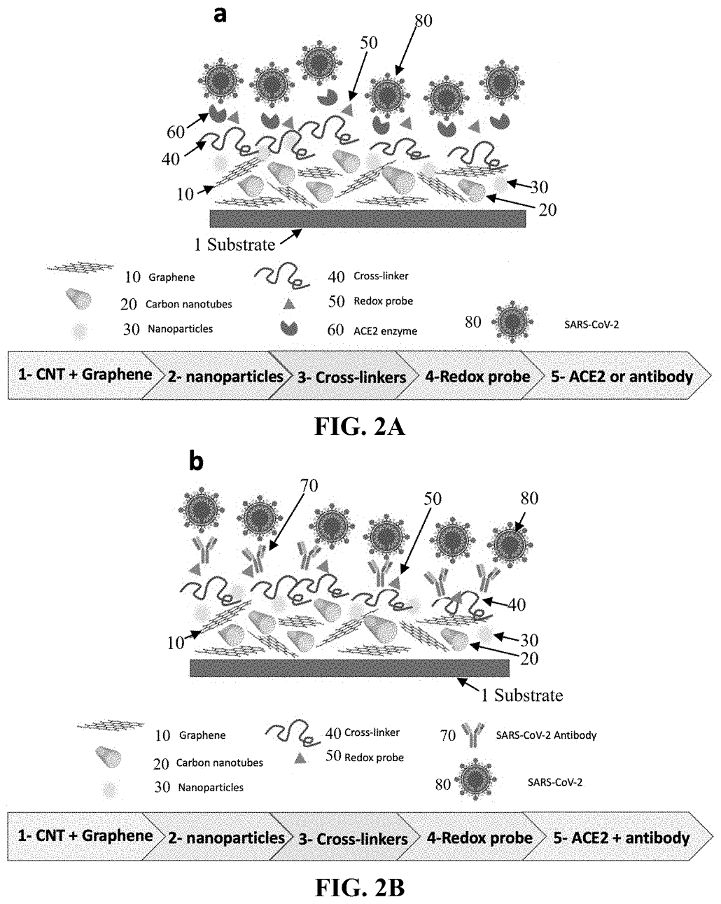

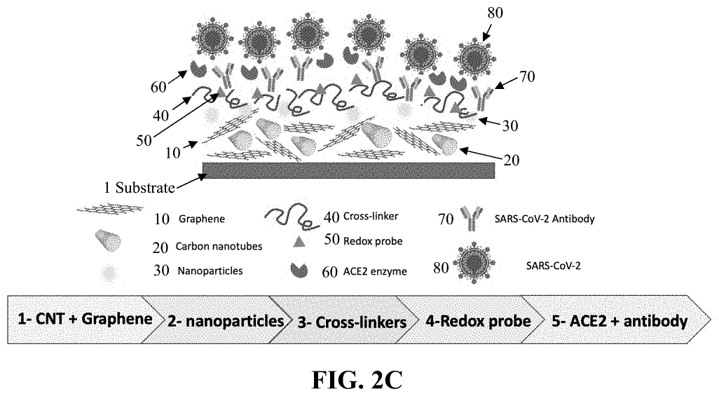

In an example of the electrode functionalization of the device, FIG. 2A depicts an ACE2 enzyme-based, SARS-CoV-2 sensor fabrication process and layers, in which a virus will only bind through one of its binding sites to ACE2. The five layers depicted at the bottom of FIG. 2A and FIG. 2B can be in any combination. A binding of SARS-CoV-2 to ACE2 is depicted at the top of FIG. 2A. In this example, ACE2 is used to detect SARS-CoV-2 specifically. A substrate is depicted by the solid bar at the bottom of FIG. 2A and at the bottom of FIG. 2B. The substrate can be an insulating layer, a conductive layer, a semi-conducting layer, or a combination thereof. For example, the substrate can include plastics, glass, polymers, silicon, stainless steel, polyimide, polyester, polydimethylsiloxane (PDMS), paper, polyethylene terephthalate, polyethylene naphthalate, cotton, poly methyl methacrylate, or a combination thereof. Examples of conductive materials can include gold, graphite, carbon, platinum, silver/silver chloride, aluminum, graphene, lead, carbon steel, nickel, chromium, copper, silver, tantalum, antimony, tin, tungsten, iridium, bismuth, titanium, molybdenum, or a combination thereof. Graphene and/or carbon nanotubes (of any thickness) can be associated with nanoparticles and one or more cross-linkers. The graphene and/or carbon nanotubes can be utilized for conduction. An ACE2 enzyme is shown associated with SARS-CoV-2 at the top of FIG. 2A. In another example, FIG. 2B depicts a SARS-CoV-2 specific antibody-based (or aptamer-based), sensor fabrication process and layers in which a virus will only bind through one of its binding sites to the antibodies or aptamers.

ACE2 can be used with a SARS-CoV-2 specific antibody and/or aptamers to reduce false negatives/positives when detecting SARS-CoV-2. An example is depicted in FIG. 2C. As shown in FIG. 2C, the combination of enzyme (ACE2) and antibody used as complementary bioreceptor in a virus sensor, in which virus will bind, through its different binding sites, to both ACE2 and antibody/aptamer, which can lead to lower risk of false negative/positive readouts.

The device can be an electrochemical device and can provide a sensor platform including one or more of the functionalization coatings depicted in FIGS. 2A, 2B, and 2C. As shown in FIGS. 2A, 2B, and 2C, a cross-linker can be used to assemble and stabilize multiple layers and/or sublayers of films. In the sensor platform an electrochemical technique of square wave voltammetry (SWV) can be performed to reach high sensitivity in detecting SARS-CoV-2. The sensor platform can detect SARS-CoV-2 within a short time (on the order of 1, 2, 3, 4, 5, or 10 minutes) with high accuracy and sensitivity. The lower limit of detection can be about 1,000, about 10,000, about 100,000, or about 1,000,000 virus particles or virus components per mL.

Fabrication of the sensor can include self-assembly multilayered films on a substrate, or on an electrode, or can include a drop coating or drop casting deposition of a suspension containing multiple components. One or more filters or semi-permeable membranes, (e.g., Nafion or a lipid bilayer membrane) optionally can be added to act as a filtration layer for filtering the liquid sample and/or also as a protection layer, for example, to enhance shelf life.

The entire sensor platform has a long shelf life, which is the length of time that the sensor remains usable (e.g., sufficiently sensitive and accurate), fit for use, or saleable. The sensor can be provided with or without external packaging. The salivary-based COVID-19 (or other viral) detection biosensor herein can have a shelf life of more than about six months, more than about a year, or more than about two years. The test sensitivity and accuracy is preserved during the storage time. In an example, the biosensor can be packaged and stored at a temperature in the range from about −20° C. to about 40° C., optionally in the range from about 0° C. to about 35° C., optionally in the range from about 5° C. to about 30° C., and optionally in the range from about 5° C. to about 30° C. Packaging can be designed to inhibit environmental humidity changes and/or exposure to electromagnetic radiation (e.g., UV light). In another example, packaging can include a sensor/indicator on the exterior of the packaging that indicates exposure to conditions that may yield the contents unsuitable for use. The sensor or packaging can include an indication of pressure, electromagnetic radiation, temperature, or other conditions encountered during storage.

Urgent need and commercial application of this technology are illustrated by the global pandemic of COVID-19. While the SARS-CoV-2 spread was first identified around December 2019, rapid detection of both symptomatic and asymptomatic individuals can be provided by this technology. Generally, air travel has significantly increased the global reach of viruses, while the technology to screen for viruses has lagged. The present technology finds application as a saliva-based POC system for viral diseases (e.g. COVID-19). The technology can be developed for home use as well as at hospitals, laboratories, clinics, health-care facilities, doctors' offices, workplaces, airports, ports of entry, and schools.

The POC sensor can detect COVID-19 through a self-collected salivary specimen. This sensor platform can include a salivary-based sensor in communication with a portable electronic meter. A microprocessor (or computer) can be included, along with display, WIFI, cloud-based storage, blue-tooth, or other electronic components. The sensor can indicate a positive result or a negative result, for ease of use.

Among all detection techniques, electrochemical methods can have an advantage over others because of their relative simplicity and inexpensiveness.^{23,24} Currently, there are various methods available to detect COVID-19, each having significant limitations. Some on-the-market methods for detecting virions in solution (including SARS-CoV-2) include Reverse-Transcription Polymerase Chain Reactions (RT-PCR) with viral cell cultures, or the newer Reverse Transcription Loop-Mediated Isothermal Amplification (RT-LAMP) for rapid detection, in which the RNA of SARS-CoV-2 is amplified to produce millions of copies in order to bring the viral concentration into a detectable range.²⁵⁻³⁰ For example, RT-PCR is used by a Rutgers University lab to detect COVID-19 through saliva in 24-72 hours with a detection limit of 200 copies/mL.³¹ However, this method suffers from its own complexity, a relatively long turnaround time, expensive equipment requirements, risk of false-negative/positive²⁵ and requires the patient to visit a medical office. RT-LAMP has overcome the turnaround time issue by utilizing 4 primers, but the other limitations still exist. Abbot has developed a RT-LAMP test to detect SARS-CoV-2 in nasopharyngeal specimen that only takes 15 minutes with a limit of detection as low as 150 copies/mL.³² However, RT-LAMP is a semi-invasive test where trained healthcare workers need to collect sample, and still requires expensive laboratory equipment. Hence it can only be done in hospital and clinics. Research in viral detection has also been done through cutting edge techniques such as Raman spectroscopy, optofluidics, interferometry, and surface plasmon resonance (SPR). These techniques are promising but suffer from the same setbacks as RT-PCR/LAMP, in that they require expensive equipment and trained personnel to perform.³³⁻³⁷ On the contrary, electrochemical methods are low-cost, simple, and fast, as well as can be done by the patient themselves, similar to how blood glucometers are used now, and have been since their inception in the 1970s as POC devices.³⁸ Over the past decade plus, there have been more and more attempts to extend electrochemical techniques in to the realm of diagnostic testing for viral diseases.³⁹⁻⁴⁴

Electrochemical sensors measure molecular concentrations by correlating them to an electrical signal using a bioreceptor and transducer. The bioreceptors are biomolecules (e.g. oligonucleotides, antibodies, or peptides) that bind to or interact with the target molecules (e.g., herein SARS-CoV-2), which then catalyze a measurable signal on the transducer. Advanced biosensors now utilize cutting edge nanotechnology to increase sensitivity and lower the limit of detection (LOD). For example, the biosensor described herein can leverage experience with DNA carbon nanotube-based sensing technologies⁴⁵⁻⁵², electrochemical salivary based POC glucose monitoring^{14,53-58}, computational screening of molecular interactions and DNA sequences,⁵⁹⁻⁶² and it can be adapted for any viral infection with modification of bioreceptors, surface binding agents, and specific linkers for each virus.

In order to detect virions using biosensors, a bioreceptor that specifically binds to intact viruses or viral proteins is required. The bioreceptor can be, for example, antibodies normally expressed by the body to fight the virus, DNA, RNA or proteins that are complementary to the viral genome/protein. Herein is provided a SARS-CoV-2 electrochemical biosensor using cutting-edge nanomaterials to measure the morphology change of the surface chemistry caused by the interaction between the bioreceptor and the surface proteins of SARS-CoV-2.

SARS-CoV-2 is composed of four target protein classes, the spike protein (S), membrane protein (M), nucleocapsid protein (N) and envelope protein (E). The genome of the virus is encapsulated inside the nucleocapsid protein, while the S protein binds to human host Angiotensin-Converting Enzyme 2 (ACE2), which allows the virus to enter human cells and begin replication.⁶³⁻⁶⁵ In this technology, the commercially available human ACE2 protein is used as a SARS-CoV-2 specific bioreceptor in the electrochemical biosensor. This POC biosensor is capable of detecting and quantifying active SARS-CoV-2 concentration in an individual's saliva sample. The ACE2 receptor binds selectively to some coronaviruses,^{66,67} and excludes other common respiratory viruses such as influenza, which further ensures that this method is able to distinguish between COVID-19 and other diseases. Furthermore, in order to accomplish the electrochemical reaction between the sensor chemistry and electrode surface, a redox-active probe such as ferrocene or methylene blue (MB) can be utilized in the fabrication chemistry. The redox-active probe has a predictable and reproducible electrochemical profile under controlled conditions without presence of target molecule in solution. In the presence of SARS-CoV-2, the morphology of the bioreceptor (i.e. ACE2) on the sensor surface changes, and results in a change in the electrochemical profile of redox-active probe. This change in the output signal (herein, current) is proportional to the amount of virus present in the sample.

In order to functionalize the aforementioned SARS-CoV-2 biosensor, the working electrode is coated with a multi-layer structure consisting of specific biomaterials as bioreceptors (ACE2/SARS-CoV-2 antibody alone or in combination as hybrid receptors or combined with SARS-CoV-2 specific aptamers), a mixture of nanomaterials (such as gold-nanoparticles, GNp), graphene, carbon-nanotubes, CNTs, or a combination thereof) in the presence of a cross-linker (such as 1-Ethyl-3-(3-dimethylaminopropyl)carbodiimide, EDC), N-hydroxy succinimide (NHS), cysteamine, 1,6-hexanedithiol, or a combination thereof). The outstanding electrical properties of carbon nanomaterials (e.g., graphene & CNT) enable ballistic transport with high electron mobility that offer unprecedented opportunities for efficient sensors.⁶⁸⁻⁷⁰ Nanoparticles can be used with carbon-based composites because they provide a suitable environment for electron transfer and biomolecule immobilization, which improve the sensor's overall sensitivity.⁷¹

The coating procedure of the proposed nanocomposite onto the sensor electrodes (working electrode alone or all three electrodes) consists of self-assembly of multi-layer films, or a one-time drop coating deposition process using a mixture solution including, but not limited to, nanomaterials, metal nanoparticles, enzymes/antibodies (alone or in combination to antibody/aptamers), cross-linkers, surfactant, blocking proteins, and redox-active probes (e.g., MB or ferrocene). Several known approaches are used to immobilize bioreceptors (herein ACE2/SARS-CoV-2 antibody alone or in combination as hybrid receptors or combined with SARS-CoV-2 specific aptamers) on biosensor chips, which can include passive conjugation,^{72,73} and/or covalent conjugation.^{74,75} The passive method is impractical due to low efficiency and stability, while covalent conjugation of bioreceptors can result in greater stability and reproducibility, hence it can provide a reliable quantification of analytes (herein SARS-CoV-2) that can hardly be achieved by passive conjugation.⁷⁶ In this technology the bioreceptor can be immobilized (e.g., ACE2/SARS-CoV-2 antibody alone or in combination as hybrid receptors or combined with SARS-CoV-2 specific aptamers) through a covalent conjugation method utilizing covalent cross-linkers. The bioreceptor to nano-composite ratio can be precisely controlled to reach to optimal bioreceptor loading and maximum sensitivity of SARS-CoV-2 detection. Moreover, the crosslinking approach, in addition to the presence of non-ionic surfactants, can stabilize the immobilized enzymes, leading to the desired expected shelf life of the sensor.^{75,58} The cross-reactivity of this sensor to other biomarkers in the sample can be reduced by adding blocking proteins (e.g. bovine serum albumin (BSA), polyethylene glycol (PEG), gelatin, in combination or including other blocking proteins) to the chemical mixture and performing a pretest saliva sample treatment (e.g. filtration, adding stabilizing agents, extracting S protein from viruses surface). The ACE2 enzyme and SARS-CoV-2 specific antibodies (e.g. S1 spike protein monoclonal antibody, CR3022) bind to different binding sites of the virus, therefore using ACE2 enzyme and an antibody in parallel, as complementary bioreceptors, in addition to a blocking protein, can reduce the risk of false negative/positive results. Redox probes are widely used as conductive substrates and redox reagents in aptamer-based biosensors.⁷⁶⁻⁸¹ Herein, it is added to the fabrication composite in order to precisely monitor the electrochemical behavior of the sensor in the presence of low concentrations of virus. Consequently, the fabrication composite and coating procedure is provided herein to realize stability and sensitivity in SARS-CoV-2 detection and reduce the required detection time to minutes, without needing any expensive equipment.

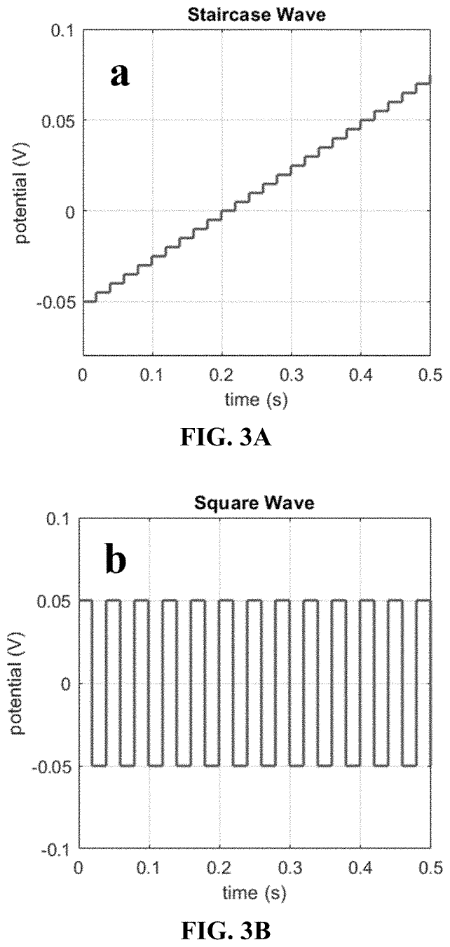

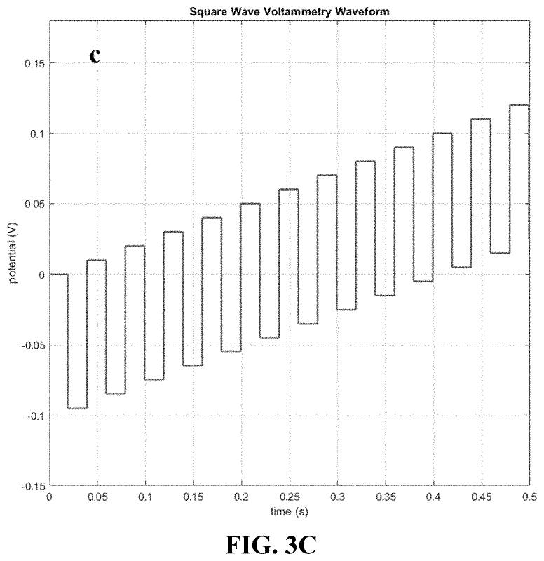

To realize the POC device, an electrochemical technique called square wave voltammetry (SWV) can be used, which is a highly sensitive alternating current (AC) voltammetric method that uses the superposition of a staircase wave and a square wave potential applied to a stationary set of electrodes as the excitation to the electrochemical cell. An example of a staircase wave component of a SWV excitement potential is depicted in FIG. 3A, where every period on the staircase is increased by a predetermined parameter Eₛₜₑₚ. An example of a square wave component of the SWV excitement potential, where the amplitude of the wave is a predetermined parameter Eₐₘₚ is depicted in FIG. 3B. The period is identical to that of the staircase wave in FIG. 3A. A combination of the component waves (staircase wave component and square wave component) that produce the square wave voltammetry excitement waveform is illustrated in FIG. 3C.

The SWV technique can be seen as a special case of Differential Pulse Voltammetry (DPV) where the duty cycle of the pulse is exactly 50%. It is considered one of the more advanced electrochemical techniques because of its utilization and superposition of a pulse technique (square wave, enhanced sensitivity), scanning technique (sweep voltammetry, insight into electrode mechanism) and impedimetric technique (kinetic information for fast electrode processes).^{24,82-84}

SARS-CoV-2 and its spike protein itself are not strictly electrochemically active, although each protein has its isoelectric point (IP) having to do with the pH of the solution. The technology does not electrochemically detect the binding of the virus to the surface. The binding of the S protein to its complimentary antibody or ACE2 does not release an electron, as it is a conformational reaction (lock and key), and the basic structures of the reactants do not change electrochemically. Therefore, direct current from binding of the virions to the surface is not measured. Instead, in the case where binding is occurring on the surface of the sensor, the surface capacitance, and efficiency of electrochemical reaction intrinsic to the sensor, is impeded. SWV is sensitive to this surface morphological change, and there will be less surface area for the redox probe imbedded in the chemistry on the surface to react with the electrolyte solution (saliva), thus the output current will decrease in proportion to the concentration of virions present. It should be noted that the density of virus receptor in the functionalization layer should be sufficiently high as to allow the binding of the virus particles to inhibit the redox reaction at the electrode; the optimum density can be determined by varying the virus receptor density.

For example, the AC nature of this technique can enable the technology to get around (cancel out) the charging currents (I_{C}) that decay as an exponential function of time (e^{-t}), and hence more quickly than the faradaic currents (I_{F}) which decay as a function of the inverse square root of time

$$( \frac{1}{\sqrt{t}} ) ,$$

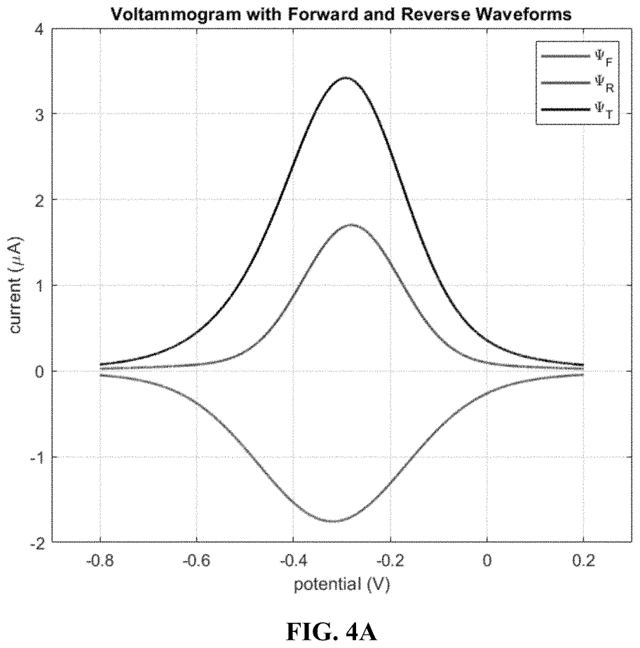

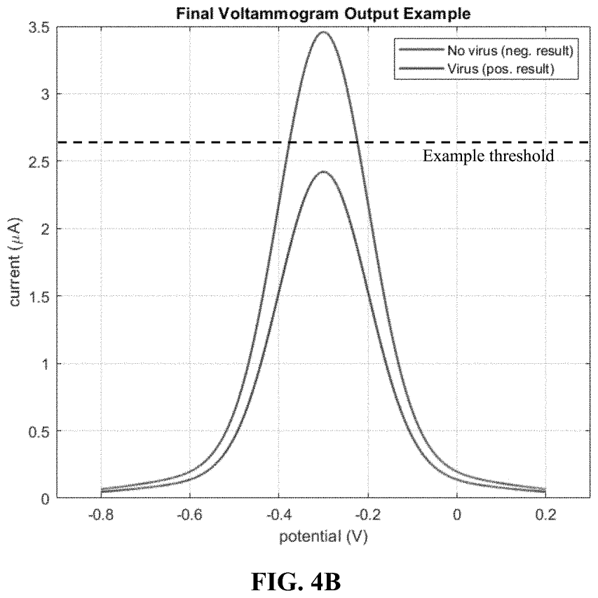

and contain the pertinent information. Therefore, the device can be configured to wait until the end of each half cycle of the excitation waveform to take the measurement, where the faradaic currents dominate. Moreover, in the area of potential activation of the specific reaction, because of the superimposed square-wave, the electrochemical cell is brought over and below the activation potential each cycle, over many cycles. Thus, multiple cycles of forward and reverse reaction can be obtained, yielding two separate forward and reverse reaction waveforms (Ψ_{F} and Ψ_{R} respectively, seen in FIG. 4A) that are then combined to give the final waveform (Ψ_{T}) such that:

Ψ_{T}=−(Ψ_{F}−Ψ_{R}).^{24,80}

An example of forward (Ψ_{F}, bottom waveform), reverse (Ψ_{R}, middle waveform) and total (Ψ_{T}, top waveform) waveforms that can make up the final voltammogram is shown in FIG. 4A. It is this summation of forward and reverse reactions that cancels out the charging currents due to the double layer capacitance, allowing the determination of the kinetics of the electrochemical reaction, that are proportional to the concentration of the analyte in solution, as shown in FIGS. 3A-3C and FIGS. 4A-4B.^{24,80-82} An example of final output voltammograms, with an example of a positive result waveform (bottom waveform) when virus is present in the solution, versus when virus is not present (negative result is top voltammogram), is depicted in FIG. 4B. In FIG. 4B, an example threshold value for positive and negative results is illustrated as the dotted horizontal line. The threshold can be established by calibration, and thereafter devices can include the threshold.

As discussed above, saliva has been shown to be clinically promising as a diagnostic tool for the detection of SARS-CoV-2 infection,^{85,86} and there have even been cases where saliva has been shown to be a more reliable marker than nasopharyngeal fluid by concentration of the virus.^{85,86} SWV and similarly DPV and electrochemical impedance spectroscopy (EIS), have been used to detect viruses such as human norovirus (HuNoV), Middle East Respiratory syndrome coronavirus (MERS-CoV) and hepatitis E virus (HEV) down to clinically relevant levels.⁴²⁻⁴⁴ Using a combination of the pulse induced method laid out by Chowdhury et al. and a specific parameter SWV for the application of this technology, SARS-CoV-2 can be detected in saliva with a detection limit of between about 1,000 and about 10,000 copies/mL (about 1.6 to about 16 attomolar, aM), which is slightly lower than what Giamberardino et al. were able to achieve when they detected HuNoV down to 20 aM, 12,000 copies/mL, and well below the reported median concentration in saliva of 3,300,000 copies/mL (5.4 fM) in patients displaying symptoms.^{44,85,86}

This technology is the first salivary-based electrochemical POC platform that is developed to accurately detect SARS-CoV-2 through self-collected saliva. In this technology, the ACE2 or SARS-CoV-2 antibody bioreceptors (alone or in combination as hybrid receptors or combined with SARS-CoV-2 specific aptamers) in addition to the cutting-edge nanomaterials are implemented to specifically detect SARS-CoV-2 using a portable electronic meter.

Example advantages and features of the present technology can include: S1 spike protein is used to detect SARS-CoV-2 specifically. ACE-2 enzyme is used to detect SARS-COV-2 specifically. Combination of S1 spike protein antibody with ACE2 Enzyme or aptamer can be used to reduce false negatives/positives when detecting SARS-CoV-2 and improve the sensitivity. This POC sensor detects COVID-19 through self-collected salivary specimen. This sensor platform can consist of a salivary-based sensor and a portable electronic meter. The fabrication procedure of the sensor includes a self-assembly of multi-layer films, or a one-time drop coating deposition of a mixture solution containing multiple components. A cross-linker is used to assemble multiple layers of films. In this sensor platform an electrochemical technique of SWV is performed to reach high sensitivity in detecting SARS-CoV-2.

This sensor platform can detect SARS-CoV-2 within 5 minutes with high accuracy and sensitivity of 2.004×10⁷ μA·mL/Copies with a limit of detection of below 10⁵ copies/mL of COVID-19 Spike Coronavirus pseudovirus. This salivary-based COVID-19 detection biosensor has a shelf life of more than six months. The test sensitivity and accuracy are preserved during storage time. Three sensors are tested for each concentration and proved good repeatability with n=3 and RSD 3%. Negative control test is done with mouse IgG antibody to evaluate the selectivity of sensor in presence of other bioreceptor to ensure that the detected signal variation is in fact due to specific binding of SARS-CoV-2 to immobilized receptors.

In order to functionalize the aforementioned POC bio-sensor for SARS-CoV detection, the working electrode (or all three electrodes) is coated with a multi-layer structure consisting of specific biomaterials (S1 Spike protein and ACE-2 enzyme alone or in combination as hybrid receptors or combined with SARS-CoV-2 specific aptamers), a mixture of nanomaterials (such as gold-nanoparticles (GNp), graphene, carbon-nanotubes, CNTs and/or other suitable materials), redox regent (such as Thionine, Methylene blue or ferrocene redox couple and/or other redox agent), and nonionic/ionic surfactant (such as Tergitol, Tritonx-100, Tween 20 and/or other surfactants) in the presence of a cross-linker (such as 1-Ethyl-3-(3-dimethyl-aminopropyl)carbodiimide (EDC), N-hydroxy succinimide (NHS), cysteamine, 1,6-hexanedithiol, and/or other suitable cross-linker). In this technology, the commercially available S1 Spike protein antibody is used as bioreceptor to specifically detect SARS-CoV-2.

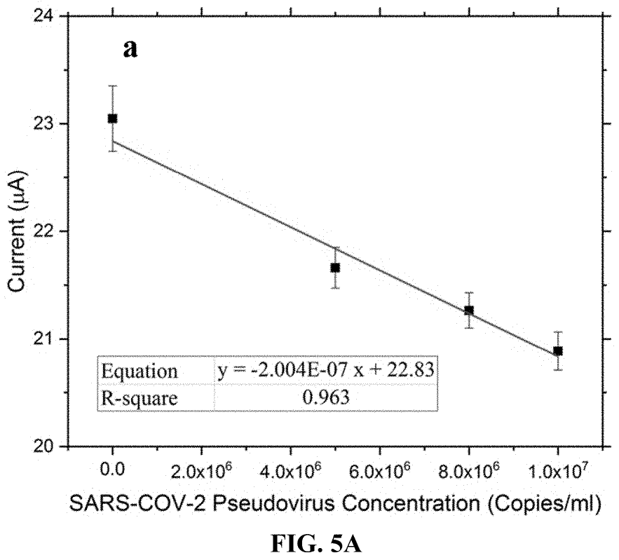

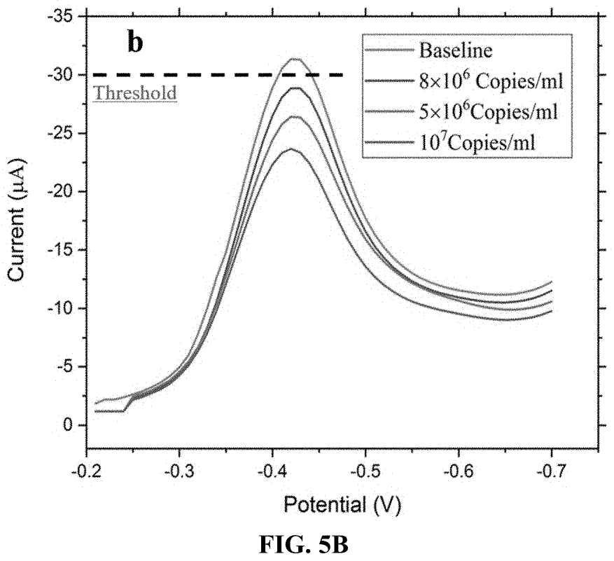

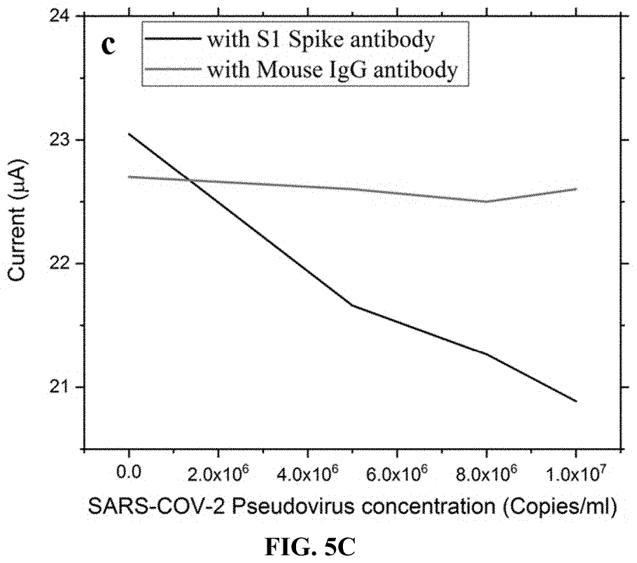

In an example, the functionalized point of care sensors are connected to a commercialized portable potentiostat to initiate the electrochemical measurement. The functionalized sensors are incubated with different concentration of COVID-19 Spike Coronavirus Pseudovirus (8×10⁶ to 1×10⁷ copies/ml) are incubated on sensors for 5 minutes. After this incubation time electrochemical SWV is performed on the modified sensor, with the output signal shown in FIG. 5B. As illustrated in FIG. 5B, with increasing the COVID-19 Spike Coronavirus Pseudovirus concentration, the output signal is reduced due to the specific binding of the virions to the immobilized receptors on surface. This binding blocks the thionine electron transfer pathway. Therefore, this change in signal can be translated to the SARS-CoV-2 concentration.

The calibration plot in FIG. 5A demonstrates a good linearity between the virus concentration and current peak height versus baseline. Within the linear range up to 5×10⁶ copies/mL with sensitivity of 2.004×10⁻⁷ μA·mL/Copies and LOD of less than 10⁵ copies/mL.

The negative control test shown in FIG. 5C is performed by immobilizing mouse IgG antibody (non-specific to SARS-CoV-2) and detecting different concentration of COVID-19 Spike Coronavirus Pseudovirus to evaluate the selectivity of sensor in presence of other bioreceptor to ensure that the trend seen in functionalized sensor is in fact due to specific binding of SARS-CoV-2 to antibody.

These results show that in presence of immobilized SARS-CoV-2 specific bioreceptors (SARS-CoV-2 antibody) the output current is significantly decreased with increasing the COVID-19 Spike Coronavirus Pseudovirus concentration which is due to the binding of the RBD part of the Pseudovirus spike protein with S1 spike protein antibody. While, in presence of mouse IgG antibody (nonspecific receptor), no significant changed in output current is observed with different concentration of COVID-19 Spike Coronavirus Pseudovirus.

This POC biosensor is capable of detecting and quantifying active SARS-CoV-2 concentrations in individual's self-collected saliva samples at a concentration below 10⁵ copies/mL. The bioreceptor (S1 Spike protein antibody) can be immobilized through a covalent conjugation method utilizing one or multiple cross-linkers (e.g. 1-Ethyl-3-(3-dimethylaminopropyl) carbodiimide (EDC), N-hydroxy succinimide (NHS), cysteamine, 1,6-hexanedithiol, etc.). Moreover, the coating composition can consist of nanomaterial matrix layer (consisting of gold-nanoparticles (GNp), graphene, carbon-nanotubes (CNTs) and/or other suitable materials) to increase the test sensitivity and lower the limit of detection. The fabrication procedure can be performed under controlled humidity (1-20%) to avoid sensor by sensor variation/unrepeatability. The functionalized sensor is packed appropriately and kept in defined environmental conditions to preserve the shelf life of the sensor.

This technology realizes a saliva-based POC biosensor capable of performing rapid, non-invasive, highly sensitive, reliable, and accurate COVID-19 test. This sensor platform has the potential to be used for other viral disease detection with minimal modifications in bioreceptor and cross-linker choice. Moreover, this point-of-care detection platform enables people to perform the viral test at home. The rapidity of the test makes it an ideal option to screen infected individuals (including those who are asymptomatic), specifically for POC screening at hospitals, laboratory, clinics and health care facilities, workplaces, ports of entries, schools, in homes, in community screening settings, or for people on the go. Furthermore, this device could be developed into an affordable home viral test kit for rural areas.

The methods and the devices described herein can include any suitable microprocessor system or suitable computing system, either with or without GPS capabilities. Software can be included operable with a processor. Processing tasks can be carried out by one or more processors or microprocessors.

An interface between the devices and the system can be provided. The computing device can include one or more input/output interfaces for connecting input and output devices to various other components of the computing device.

While this technology has been described with references to example embodiments thereof, it will be understood by those skilled in the field that various changes in form and details may be made therein without departing from the scope of the technology encompassed by the appended claims.

### EXAMPLES

### Example 1. Fabrication of Biosensor for Detection of SARS-CoV-2

Pseudovirus containing S1 spike protein of SARS-CoV-2 was obtained from MyBioSource. Antibody against SARS-CoV-2 S1 spike was obtained from Genscript.

A commercially available electrochemical sensor with bare electrodes on a substrate was cut and washed with DI water. The washed sensor was stored in a desiccator for 10 minutes before starting the fabrication to ensure the sensor was fully dried.

A suspension of carbon nanotubes (150-170 mg/ml) was mixed with Tergitol (1% by wt), and the suspension was sonicated at room temperature. The mixed suspension was drop cast on the working electrode, and the sensor put in a desiccator until fully dried.

A suspension of gold nanoparticles was mixed with thionine (2 mg/ml) at room temperature. The suspension was then drop cast on the sensor, which was again dried in a dessicator.

A solution of S1 spike protein antibody was prepared in PBS solution and then drop cast onto the sensor, which was fully dried in a dessicator.

Finally, sensors were placed into a sealed, dark gel box, which was stored at 4° C. until use.

### Example 2. Detection of SARS-CoV-2 Pseudovirus

In this Example, the commercially available S1 spike protein antibody was used as bioreceptor to specifically detect SARS-CoV-2 pseudovirus. The functionalized point of care sensor was connected to a commercial portable potentiostat to initiate the electrochemical measurement. The functionalized sensor was incubated with different concentrations of pseudovirus, (8×10⁶ to 1×10⁷ copies/mL) which was then incubated on the sensors for 5 minutes. After this incubation time, electrochemical square wave voltammetry (SWV) was performed on the modified sensor, and the output signal is shown in FIG. 5B. With increasing pseudovirus concentration, the output signal was reduced due to the specific binding of the pseudovirus particles to the immobilized receptors on the functionalized working electrode surface. This binding blocked the thionine electron transfer pathway. Therefore, the change in signal could be related to the SARS-CoV-2 concentration.

A calibration plot shown in FIG. 5A demonstrated a linear relation between the virus concentration and current peak height versus baseline. Within the linear range, up to 5×10⁶ copies/mL could be detected with a sensitivity of 2.004×10⁻⁷ μA·mL/copy and a LOD of less than 10⁵ copies/mL.

### Example 3. Negative Control Test

The negative control test shown in FIG. 5C was performed by immobilizing a mouse IgG antibody (non-specific with respect to SARS-CoV-2) in a sensor functionalization coating instead of a SARS-CoV-2 S1-specific antibody and testing different concentration of SARS-CoV-2 pseudovirus. This allowed evaluation of the selectivity of the sensor. The sensor containing immobilized SARS-CoV-2 specific antibody showed a significant decrease in the output current with increasing pseudovirus concentration, due to binding of the RBD part of the pseudovirus spike protein with the S1 antibody. However, when the S1 antibody was substituted with mouse IgG antibody with no binding specificity for the psuedovirus, no significant change in output current was observed at any tested concentration of pseudovirus.

### REFERENCES

1. Mortimer, Philip P., and John V. Parry. “Detection of antibody to HIV in saliva: a brief review.” Clinical and diagnostic virology 2.4-5 (1994): 231-243.
2. Oba, Isabel Takano, et al. “Detection of hepatitis A antibodies by ELISA using saliva as clinical samples.” Revista do Instituto de Medicina Tropical de São Paulo 42.4 (2000): 197-200.
3. Arora, G., et al. “Saliva as a tool in the detection of hepatitis B surface antigen in patients.” Compendium of continuing education in dentistry (Jamesburg, N.J.: 1995) 33.3 (2012): 174-6.
4. González, V., et al. “Detection of hepatitis C virus antibodies in oral fluid specimens for prevalence studies.” European Journal of Clinical Microbiology & Infectious Diseases 27.2 (2008): 121-126.
5. Sosa-Jurado, Francisca, et al. “Detection of hepatitis C virus RNA in saliva of patients with active infection not associated with periodontal or liver disease severity.” BMC infectious diseases 14.1 (2014): 72.
6. Mager, D. L., et al. “The salivary microbiota as a diagnostic indicator of oral cancer: a descriptive, non-randomized study of cancer-free and oral squamous cell carcinoma subjects.” Journal of translational medicine 3.1 (2005): 27.
7. Khurshid, Zohaib, et al. “Role of salivary biomarkers in oral cancer detection.” Advances in clinical chemistry. Vol. 86. Elsevier, 2018. 23-70.
8. Sugimoto, Masahiro, et al. “Capillary electrophoresis mass spectrometry-based saliva metabolomics identified oral, breast and pancreatic cancer-specific profiles.” Metabolomics 6.1 (2010): 78-95.
9. Streckfus, Charles F., Lenora R. Bigler, and Michael Zwick. “The use of surface-enhanced laser desorption/ionization time-of-flight mass spectrometry to detect putative breast cancer markers in saliva: a feasibility study.” Journal of oral pathology & medicine 35.5 (2006): 292-300.
10. Asai, Yasutsugu, et al. “Elevated polyamines in saliva of pancreatic cancer.” Cancers 10.2 (2018): 43.
11. Qian, Kun, et al. “New method of lung cancer detection by saliva test using surface-enhanced Raman spectroscopy.” Thoracic cancer 9.11 (2018): 1556-1561.
12. Xiao, Hua, et al. “Proteomic analysis of human saliva from lung cancer patients using two-dimensional difference gel electrophoresis and mass spectrometry.” Molecular & Cellular Proteomics 11.2 (2012).
13. Foley I I I, Joseph D., et al. “Oral fluids that detect cardiovascular disease biomarkers.” Oral surgery, oral medicine, oral pathology and oral radiology 114.2 (2012): 207-214.
14. Du, Yunging, Wenjun Zhang, and Ming L. Wang. “An on-chip disposable salivary glucose sensor for diabetes control.” Journal of diabetes science and technology 10.6 (2016): 1344-1352.
15. Reynolds S J, Muwonga J. OraQuick ADVANCE Rapid HIV-1/2 antibody test. Expert Rev Mol Diagn. 2004; 4(5):587-591
16. Khurshid, Zohaib, et al. “Human saliva canbe a diagnostic tool for Zika virus detection.” Journal of infection and public health (2019).
17. Barzon, Luisa, et al. “Isolation of infectious Zika virus from saliva and prolonged viral RNA shedding in a traveller returning from the Dominican Republic to Italy, January 2016.” Eurosurveillance 21.10 (2016): 30159.
18. Wang, Wei-Kung, et al. “Detection of SARS-associated coronavirus in throat wash and saliva in early diagnosis.” Emerging infectious diseases 10.7 (2004): 1213-.
19. Wang, Wei-Kung, et al. “Detection of SARS-associated coronavirus in throat wash and saliva in early diagnosis.” Emerging infectious diseases 10.7 (2004): 1213-1219.
20. Kim, Young-Il, et al. “Infection and rapid transmission of SARS-CoV-2 in ferrets.” Cell host & microbe (2020).
21. To, K. K. W., et al. “Saliva as a diagnostic specimen for testing respiratory virus by a point-of-care molecular assay: a diagnostic validity study.” Clinical Microbiology and Infection 25.3 (2019): 372-378.
22. “Coronavirus (COVID-19) Update: FDA Authorizes First Diagnostic Test Using At-Home Collection of Saliva Specimens” U.S. Food and Drug Administration, 18 May 2020. Press release.
23. Brownson, Dale A C, and Craig E. Banks. “Interpreting electrochemistry.” The Handbook of Graphene Electrochemistry. Springer, London, 2014. 23-77.
24. Mirceski, Valentin, Sebojka Komorsky-Lovric, and Milivoj Lovric. Square-wave voltammetry: theory and application. Springer Science & Business Media, 2007.
25. Tahamtan, Alireza, and Abdollah Ardebili. “Real-time RT-PCR in COVID-19 detection: issues affecting the results.” (2020): 453-454.
26. David, D., et al. “Rabies virus detection by RT-PCR in decomposed naturally infected brains.” Veterinary microbiology 87.2 (2002): 111-118.
27. Schwab, Kellogg J., et al. “Distribution of Norwalk virus within shellfish following bioaccumulation and subsequent depuration by detection using RT-PCR.” Journal of food protection 61.12 (1998): 1674-1680.
28. Spackman, Erica, and David L. Suarez. “Type A influenza virus detection and quantitation by real-time RT-PCR.” Avian influenza virus. Humana Press, 2008. 19-26.
29. Komminoth, P., et al. “Evaluation of methods for hepatitis C virus detection in archival liver biopsies: comparison of histology, immunohistochemistry, in-situ hybridization, reverse transcriptase polymerase chain reaction (RT-PCR) and in-situ RT-PCR.” Pathology-Research and Practice 190.11 (1994): 1017-1025.
30. Pham, Hang Minh, et al. “Loop-mediated isothermal amplification for rapid detection of Newcastle disease virus.” Journal of clinical microbiology 43.4 (2005): 1646-1650.
31. Food, U. S. “Drug Administration. Accelerated Emergency Use Authorization (EUA) Summary SARS-CoV-2 Assay (Rutgers Clinical Genomics Laboratory).”
32. Carter, Linda J., et al. “Assay techniques and test development for COVID-19 diagnosis.” (2020): 591-605.
33. Cialla, Dana, et al. “Raman to the limit: tip-enhanced Raman spectroscopic investigations of a single tobacco mosaic virus.” Journal of Raman Spectroscopy: An International Journal for Original Work in all Aspects of Raman Spectroscopy, Including Higher Order Processes, and also Brillouin and Rayleigh Scattering 40.3 (2009): 240-243.
34. Vollmer, F., S. Arnold, and D. Keng. “Single virus detection from the reactive shift of a whispering-gallery mode.” Proceedings of the National Academy of Sciences 105.52 (2008): 20701-20704.
35. Ozcelik, Damla, et al. “Optofluidic wavelength division multiplexing for single-virus detection.” Proceedings of the National Academy of Sciences 112.42 (2015): 12933-12937.
36. Ymeti, Aurel, et al. “Fast, ultrasensitive virus detection using a young interferometer sensor.” Nano letters 7.2 (2007): 394-397.
37. Wang, Shaopeng, et al. “Label-free imaging, detection, and mass measurement of single viruses by surface plasmon resonance.” Proceedings of the National Academy of Sciences 107.37 (2010): 16028-16032.
38. Clarke, S. F., and J. R. Foster. “A history of blood glucose meters and their role in self-monitoring of diabetes mellitus.” British journal of biomedical science 69.2 (2012): 83-93.
39. Mahari, Subhasis, et al. “eCovSens-Ultrasensitive Novel In-House Built Printed Circuit Board Based Electrochemical Device for Rapid Detection of nCovid-19 antigen, a spike protein domain 1 of SARS-CoV-2.” bioRxiv (2020).
40. Nidzworski, Dawid, et al. “A rapid-response ultrasensitive biosensor for influenza virus detection using antibody modified boron-doped diamond.” Scientific reports 7.1 (2017): 1-10.
41. Sepunaru, Lior, et al. “Rapid electrochemical detection of single influenza viruses tagged with silver nanoparticles.” Chemical science 7.6 (2016): 3892-3899.
42. Chowdhury, Ankan Dutta, et al. “Electrical pulse-induced electrochemical biosensor for hepatitis E virus detection.” Nature communications 10.1 (2019): 1-12.
43. Layqah, Laila Ali, and Shimaa Eissa. “An electrochemical immunosensor for the corona virus associated with the Middle East respiratory syndrome using an array of gold nanoparticle-modified carbon electrodes.” Microchimica Acta 186.4 (2019): 224.
44. Giamberardino, Amanda, et al. “Ultrasensitive norovirus detection using DNA aptasensor technology.” PloS one 8.11 (2013).
45. Liu, Y., et al. “The effect of sequence length on DNA decorated CNT gas sensors.” 2011 16th International Solid-State Sensors, Actuators and Microsystems Conference. IEEE, 2011.
46. Khamis, Samuel M., et al. “Homo-DNA functionalized carbon nanotube chemical sensors.” Journal of Physics and Chemistry of Solids 71.4 (2010): 476-479.
47. Zhang, Wenjun, Yu Liu, and Ming L. Wang. “DNA-functionalized single-walled carbon nanotube-based sensor array for gas monitoring.” Smart Structures and Systems 12.1 (2013): 73-95.
48. Liu, Yu, et al. “Carbon nanotube sensors integrated inside a microfluidic channel for water quality monitoring.” Sensors and Smart Structures Technologies for Civil, Mechanical, and Aerospace Systems 2011. Vol. 7981. International Society for Optics and Photonics, 2011.
49. Liu, Yu, et al. “DNA decorated carbon nanotube sensors on CMOS circuitry for environmental monitoring.” Sensors and Smart Structures Technologies for Civil, Mechanical, and Aerospace Systems 2010. Vol. 7647. International Society for Optics and Photonics, 2010.
50. Liu, Yu, et al. “SWNT based nanosensors for wireless detection of explosives and chemical warfare agents.” IEEE Sensors Journal 13.1 (2012): 202-210.
51. G-Y. Liu, M. Chen, M. L. Wang, M. R. Dokmeci. RNA Functionalized SWNT Nano Devices for Chemical Sensing. Applied Physics Letters, Vol. 103, 10103 (2013).
52. Wenjun Zhang, Ming L. Wang. DNA-Functionalized Single-Walled Carbon Nanotubes-Based Sensor Array for Breath Analysis. International Journal of Electronics and Electronical Engineering 4 (2016) 177-180.
53. Wenjun Zhang, Yunqing Du, Ming L. Wang. On-chip Highly sensitive saliva glucose sensing using multilayer films composed of single-walled carbon nanotubes, gold nanoparticles, and glucose oxidase. Sensing and Bio-Sensing Research, 2015, 4, 96-102.
54. Wenjun Zhang, Yunqing Du, Ming L. Wang. Noninvasive glucose monitoring using saliva nano-biosensor, Sensing and Bio-Sensing Research, 2015, 10.1016.
55. Yunqing Du, Wenjun Zhang, Ming L. Wang. Sensing of salivary Glucose Using Nano-Structured Biosensors, Biosensors, 6(1), 2016, 10.
56. Yunging Du, Ming L. Wang. State of the art and new perspectives for non-invasive point-of-care testing, International Journal of Biosensors & Bioelectronics, 1(1), 2016, 00002. DOI: 10.15406/ijbsbe.2016.01.00002.
57. Saliva Glucose Monitoring System, with Wenjun Zhang, U.S. Pat. No. 9,244,035 B2, Jan. 26,2016. Licensed to NanoBio LLC. December 2017.
58. Yunging, Du and Ming L. Wang; PCT/US2017/065624; “Durable Enzyme-Based Biosensor and Process for Drop Deposition Immobilization”, December 2017. Licensed to SalivaTek Inc.
59. Zhang, W., Ming L. Wang, Khalili S, Cranford S W. Materiomics for Oral Disease Diagnostics and Personal Health Monitoring: Designer Biomaterials for the Next Generation Biomarkers. Omics-a Journal of Integrative Biology, 2016. 20(1): pp. 12-29.
60. Zhang, W., M. L. Wang, and S. W. Cranford. Ranking of Molecular Biomarker Interaction with Targeted DNA Nucleobases via Full Atomistic Molecular Dynamics. Nature, Sci Rep, 2016. 6: p. 18659.
61. Zhang, W., Ming L. Wang, Steven W. Cranford. Biosensor Design through Molecular Dynamics Simulation. World Academy of Science, Engineering and Technology, International Journal of Biological, Biomolecular, Agricultural, Food and Biotechnological Engineering, 2015. 10(1): p. 10-14.
62. Wenjun Zhang, Sheyda Nazarine, M. Wang, S. Cranford, “Sensor Design via Scaled Collision Theory”, ASCE Journal of Engineering Mechanics. DOI: 10.1061/(ASCE)EM.1943-7889.0001487. June 2018.
63. Wenhui Li, Michael J. Moore, Natalya Vasilieva, Jianhua Sui, Swee Kee Wong, Michael A. Berne, Mohan Somasundaran, John L. Sullivan, Katherine Luzuriaga, Thomas C. Greenough, Hyeryun Choe, Michael Farzan; Angiotensin-converting enzyme 2 is a functional receptor for SARS coronavirus. Nature 2003;
64. Hannah Balfour. 2020. Scientists demonstrate how COVID-19 infects human cells.
65. Xu X, Chen P, Wang J, et al. Evolution of the novel coronavirus from the ongoing Wuhan outbreak and modeling of its spike protein for risk of human transmission. Life Sciences 2020; 63(3):457-460.
66. Kuba K, Imai Y, Rao S, et al. A crucial role of angiotensin converting enzyme 2 (ACE2) in SARS coronavirus-induced lung injury. Nat Med. 2005; 11(8):875-879. doi:10.1038/nm1267
67. Tai, W., He, L., Zhang, X. et al. Characterization of the receptor-binding domain (RBD) of 2019 novel coronavirus: implication for development of RBD protein as a viral attachment inhibitor and vaccine. Cell Mol Immunol (2020). https://doi.org/10.1038/s41423-020-0400-4.
68. Adhikari, B.-R., M. Govindhan, and A. J. S. Chen, Carbon nanomaterials based electrochemical sensors/biosensors for the sensitive detection of pharmaceutical and biological compounds. 2015. 15(9): p. 22490-22508.
69. Vahid, Nadia Farsaei, et al. “Effect of surfactant type on buckypaper electrochemical performance.” Micro & Nano Letters 13.7 (2018): 927-930.
70. Vahid, N. F., Marvi, M. R., Naimi-Jamal, M. R., Naghib, S. M., & Ghaffarinejad, A. (2019). X-Fe204-Buckypaper-Chitosan Nanocomposites for Nonenzymatic Electrochemical Glucose Biosensing. Anal. Bioanal. Electrochem, 11, 930-942.
71. Kumar, S., et al., Electrochemical sensors and biosensors based on graphene functionalized with metal oxide nanostructures for healthcare applications. 2019. 4(18): p. 5322-5337.
72. Sharma, S., Byrne, H., & O'Kennedy, R. J. (2016). Antibodies and antibody-derived analytical biosensors. Essays in biochemistry, 60(1), 9-18.
73. Nguyen, H. H., Lee, S. H., Lee, U. J., Fermin, C. D., & Kim, M. (2019). Immobilized enzymes in biosensor applications. Materials, 12(1), 121.
74. Mazaheri, M., Simchi, A., & Aashuri, H. (2018). Enzymatic biosensing by covalent conjugation of enzymes to 3D-networks of graphene nanosheets on arrays of vertically aligned gold nanorods: Application to voltammetric glucose sensing. Microchimica Acta, 185(3), 178.
75. Li, Z., & Chen, G. Y. (2018). Current conjugation methods for immunosensors. Nanomaterials, 8(5), 278.
76. Kim, D., & Herr, A. E. (2013). Protein immobilization techniques for microfluidic assays. Biomicrofluidics, 7(4), 041501.
77. Double-do protein cross-linkers handbook and selection guide. 2016.
78. Mao, K., Wu, D., Li, Y., Ma, H., Ni, Z., Yu, H., . . . & Du, B. (2012). Label-free electrochemical immunosensor based on graphene/methylene blue nanocomposite. Analytical biochemistry, 422(1), 22-27.
79. Dutta, G., Nagarajan, S., Lapidus, L. J., & Lillehoj, P. B. (2017). Enzyme-free electrochemical immunosensor based on methylene blue and the electro-oxidation of hydrazine on Pt nanoparticles. Biosensors and Bioelectronics, 92, 372-377.
80. Safaei, M., Beitollahi, H., & Shishehbore, M. R. (2020). Detection of Cytochrome C Using a Selective and Sensitive Methylene Blue-Based Electrochemical Aptasensor. Fine Chemical Engineering, 1-8.
81. Raouafi, A., Sánchez, A., Raouafi, N., & Villalonga, R. (2019). Electrochemical aptamer-based bioplatform for ultrasensitive detection of prostate specific antigen. Sensors and Actuators B: Chemical, 297, 126762.
82. Mirceski, Valentin, et al. “Square-wave voltammetry: A review on the recent progress.” Electroanalysis 25.11 (2013):2411-2422.
83. Ramaley, Louis, and Matthew S. Krause. “Theory of square wave voltammetry.” Analytical Chemistry 41.11 (1969): 1362-1365.
84. Osteryoung, Janet G., and Robert A. Osteryoung. “Square wave voltammetry.” Analytical Chemistry 57.1 (1985): 101-110.
85. Azzi, Lorenzo, et al. “Saliva is a reliable tool to detect SARS-CoV-2.” Journal of Infection (2020).
86. Iwasaki, Sumio, et al. “Comparison of SARS-CoV-2 detection in nasopharyngeal swab and saliva.” Journal of Infection (2020).
87. To, Kelvin Kai-Wang, et al. “Consistent detection of 2019 novel coronavirus in saliva.” Clinical Infectious Diseases (2020).
88. Azzi, Lorenzo, et al. “Two cases of COVID-19 with positive salivary and negative pharyngeal or respiratory swabs at hospital discharge: a rising concern.” Oral Diseases (2020).

1. An electrochemical sensor device for detection of a virus in a fluid sample, the device comprising:
a substrate;
a working electrode and a counter electrode disposed on the substrate;
a sample placement area on the substrate, the sample placement area configured for deposition of a fluid sample into the sample placement area, wherein the deposited fluid sample covers the working and counter electrodes;
one or more functionalization layers deposited on a surface of the working electrode, the surface configured for exposure to the fluid sample deposited in the sample placement area; wherein each functionalization layer comprises one or more sensor elements, metallic nanoparticles, a crosslinker, a redox reagent, and a virus receptor.

2. The electrochemical sensor device of claim 1, wherein the device comprises from 2 to about 10 stacked functionalization coating layers.

3. The electrochemical sensor device of claim 1, wherein the one or more sensor elements, metallic nanoparticles, polymer, redox reagent, and virus receptor of each functionalization layer are disposed in separate sublayers of the functionalization layers.

4. The electrochemical sensor device of claim 3, wherein the sublayers of each functionalization layer are disposed in the order (from electrode surface upwards): sensor elements, metallic nanoparticles, crosslinker, redox reagent, virus receptor.

5. The electrochemical sensor device of claim 1, wherein the one or more sensor elements, metallic nanoparticles, polymer, redox reagent, and virus receptor of each functionalization layer are present in a single continuous functionalization layer.

6. The electrochemical sensor device of claim 5, wherein the one or more functionalization layers are each produced by drop casting.

7. The electrochemical sensor device of claim 1, wherein the sensor elements comprise a material selected from the group consisting of carbon nanotubes, graphene, graphene oxide, fullerene, buckypaper, graphite, carbon nanofibers, carbon nanowires, carbon nanopowder, nanorods, quantum dots, and mixtures thereof.

8. The electrochemical sensor device of claim 7, wherein the sensor elements comprise a mixture of carbon nanotubes and graphene.

9. The electrochemical sensor device of claim 1, wherein the metallic nanoparticles comprise a metal selected from the group consisting of gold, silver, platinum, palladium, iron, iron oxide, copper, zinc, titanium, rhodium, ruthenium, rhenium, and mixtures thereof.

10. The electrochemical sensor device of claim 1, wherein the crosslinker is selected from the group consisting of NHS.EDC, streptavidin, 1,6-hexanedithiol, glutaraldehyde, cysteamine, alkanethiol, protein A, 3-aminopropyltriethoxysilane (APTES), sulfosuccinimidyl 4-(N-maleimidomethyl) cyclohexane-1-carboxylate), dithiobis (succinimidyl propionate) (DSP), sulfhydryl-reactive (maleimide), carboxy-PEG-amine or -carboxylic acid, carboxy-PEG-lipoamide (bidentate thiol), carboxy-PEG-thiol or -carboxylic acid, methyl-PEG-amine, methyl-PEG-lipoamide (bidentate thiol), Protein G, disuccinimidyl suberate (DSS), and combinations thereof.

11. The electrochemical sensor device of claim 1, wherein the redox reagent is selected from the group consisting of methylene blue, thionine, ferrocyanide/ferricyanide, horseradish peroxidase, glucose oxidase, alkaline phosphatase, rrease, β-galactosidase, and combinations thereof.

12. The electrochemical sensor device of claim 1, further comprising a reference electrode disposed in the sample placement area, wherein the deposited fluid sample covers the reference electrode.

13. The electrochemical sensor device of claim 1, wherein the virus receptor is selected from the group consisting of cell surface proteins and fragments and variants thereof, antibodies and fragments thereof, aptamers, and combinations thereof.

14. The electrochemical sensor device of claim 1, wherein the device is configured for detecting a virus selected from the group consisting of HIV-1, HIV-2, Zika virus (ZIKV), SARS-CoV, SARS-CoV-2, influenza virus, hepatitis virus, Ebola, and combinations thereof.

15. The electrochemical sensor device of claim 14, wherein the virus is SARS-CoV-2.

16. The electrochemical sensor device of claim 15, wherein the virus receptor is selected from the group consisting of antibodies and fragments thereof binding to SARS-CoV-2 spike protein, envelope protein, RBD protein, or nucleocapsid protein; angiotensin-converting enzyme 2 (ACE2), SARS-CoV-2 specific aptamers, CR3022 antibody, CR3009 antibody, CR0018 antibody, and combinations thereof.

17. The electrochemical sensor device of claim 1, wherein the device is configured for use in performing an electrochemical measurement comprising square wave voltammetry (SWV), amperometry, cyclic voltammetry (CV), or electrochemical impedance spectroscopy (EIS).

18. The electrochemical sensor device of claim 1, wherein the device is configured for detecting a virus in a fluid sample selected from the group consisting of saliva, urine, tears, sweat, blood, and interstitial fluid.

19. The electrochemical sensor device of claim 1, wherein the device is capable of detecting virus in a fluid sample down to a virus concentration of at least about 10⁴ copies/mL.

20. A system for electrochemical detection of a virus, the system comprising the electrochemical sensor device of claim 1, a power source capable of supplying a desired voltage signal to the electrodes of the device, and a detection circuit capable of detecting current between electrodes of the device.
